(19) United States
(12) Patent Application Publication
(10) Pub. No.: US 2009/0076337 A1
(43) Pub. Date: Mar. 19, 2009

(54) Control System For Childbirth And Method Thereof

(75) Inventors: Yun Seok Yang, Daejeon (KR); Hee In Choi, Daejeon (KR)

Correspondence Address: HYUN JONG PARK;Park & Associates IP Law LLC, 265 Bic Drive, Suite 106, Milford, CT, 06461, US

(73) Assignee: Yun Seok Yang, Seo-gu (KR)

(21) Appl. No.: 12/274,562

(22) Filed: Nov. 20, 2008

(30) Foreign Application Priority Data
May 4, 2006 (KR) PCT/KR2007/002504
May 24, 2006 (KR) 10-2006-0046665
May 24, 2006 (KR) 10-2006-0046666

(51) Int. Cl. A61B 5/00

(52) U.S. Cl. 600/300

(57) ABSTRACT

A childbirth control system comprises: a plurality of PDAs (personal digital assistants) 10 for obtaining physical information of a woman in childbed and a fetus through a plurality of diagnosis devices electrically connected thereto in real-time so as to build a personal childbirth process information database; a childbirth server 20 for comparing the personal childbirth process information data obtained from each PDA (personal digital assistants) 10 in real-time through a wire/wireless communication network with a stored basic childbirth process information data and retransmitting the analyzed data to the corresponding PDA (personal digital assistants) 10 so as to set any one childbirth method of a natural childbirth and a artificial childbirth; and a plurality of monitoring terminals 30 for outputting the personal childbirth process information data obtained from each PDA (personal digital assistants) 10 in real-time through the wire/wireless communication network and the progress of the childbirth with a sound or image in order that the woman in childbed and collaborating obstetrician can visually make out them.

### REFERENCE TO RELATED APPLICATIONS

This a continuation of pending International Patent Application PCT/KR2007/002504 filed on May 24, 2007, which designates the United States and claims priority of Korean Patent Applications No. 10-2006-0046665, filed on May 24, 2006, and No. 10-2006-0046666, filed on May 24, 2006, the entire contents of which are incorporated herein by reference.

### FIELD OF THE INVENTION

The present invention relates to a childbirth control system and a method thereof, and more particularly to a childbirth control system and a method thereof capable of giving a remote medical treatment in real-time through a communication network and selecting a childbirth method according to a physical change of the woman in childbed and the fetus.

More concretely, the present invention relates to a childbirth control system and a method thereof includes a plurality of PDAs (personal digital assistants) for obtaining physical information of a woman in childbed and a fetus through a plurality of diagnosis devices electrically connected thereto in real-time so as to build a personal childbirth process information database; a childbirth server for comparing the personal childbirth process information database obtained from each PDA (personal digital assistants) in real-time through a wire/wireless communication network with a stored basic childbirth process information database and retransmitting the analyzed data to the corresponding PDA (personal digital assistants) so as to set any one childbirth method of a natural childbirth and a artificial childbirth; and a plurality of monitoring terminals for outputting the personal childbirth process information database obtained from each PDA (personal digital assistants) in real-time through the wire/wireless communication network and the progress of the childbirth with a sound or image in order that the woman in childbed and collaborating obstetrician can visually make out them.

### BACKGROUND OF THE INVENTION

Generally, there are various kinds in childbirth methods. As well known, there are a normal childbirth of progressing the childbirth satisfactorily with healthy woman in childbed and fetus and an abnormal childbirth of progressing the childbirth unsatisfactorily with dangerous woman in childbed and fetus.

Also, the normal childbirth can divide into a natural childbirth and an artificial labor according to yes or no of an artificial assistance.

In the meantime, in the progress of the childbirth, three are many requirements. For example, there are the physical conditions of the woman in childbed and fetus for normal childbirth. Where there is something wrong with the conditions, the obstetrician take proper action against the dangerous elements based on the experience and knowledge thereof.

Recently, in order to take prompt action on the dangerous elements in the course of the childbirth, many techniques and equipments have been developed, thereby reducing the medical accidents largely.

However, as though many techniques and equipments have been developed, the childbirth accidents have been occasionally occurred owing to an improper counterplan of the obstetrician and so on according to circumstances.

That is, the obstetricians can have many different opinions in terms of childbirth philosophy and management policy of the childbirth treatment in comparison with another medical field.

Especially, there is a sudden dilemma during the childbirth treatment. That is, where a complication arises in the woman in childbed and fetus, the protector has a doubt about it and after all he can be faced with a more difficult question.

There are three problems in the conventional childbirth control method.

Firstly, since there are various definitions in the childbirth progress and treatment, some childbirth treatment can be often taken for an abnormal medical treatment.

Secondly, since the childbirth is successively performed in real-time, it has difficulty in diagnosing and treating in cooperation with another obstetrician in real-time.

Thirdly, it is necessary to scientifically predict and the progress of the childbirth in a moment. However, there is no scientific data and clinical devices for this.

Especially, since the predicting ability of a danger record is not perfect, the woman of a low danger can be delivered of a baby with difficulty. On the contrary, the woman of a high danger can be safely delivered of a baby.

In this manner, in the course of the childbirth, a normal childbirth can be changed to an abnormal one or an abnormal childbirth can be changed to a normal one frequently.

Accordingly, there are various progresses of the childbirth and medical treatments thereof. Because of various definitions on the childbirth, it has difficulty in inducing a tailored type childbirth method capable of overcoming a difficult delivery and so on.

Under this circumstance, where the childbirth accidents are occurred, the doubt of the mass gets serious. Also, it becomes ill spoken of the medical expertise.

Also, the obstetrician has a tendency to adhere to his childbirth philosophy differently with other medical fields. Moreover, it is necessary to widely make a study of various definitions on childbirth and efficiency on childbirth policies and so on. However, actually, there is no actual study owing to some problems such as data unification or policy harmony and so on. Accordingly, presently, the obstetricians perform the childbirth and the diagnosis and medical treatment thereof based on the medical experience and personal opinion thereof. Therefore, the objective and scientific childbirth management system capable of implementing and treating the demands on various childbirth policies has been demanded.

In order to implement the objective and scientific childbirth management system, the collaborating medical treatment with other obstetrician is essential for the childbirth. Accordingly, instruments using an internet communication network capable of managing the childbirth according to a consistent philosophy and policy on various definitions of the childbirth and displaying the conditions of the progress of the childbirth are required. Also, it is necessary for the obstetrician to be adequately communicated with the woman in childbed, the protector thereof, and the collaborating obstetrician on the information of the progress of childbirth without remaking out the note on the progress of the childbirth and the other documents.

### SUMMARY OF THE INVENTION

Accordingly, the present invention has been made to solve the above-mentioned problems occurring in the prior art, and an object of the present invention is to provide a childbirth control system and a method thereof capable of giving a remote medical treatment in real-time through a communication network and selecting a childbirth method according to a physical change of the woman in childbed and the fetus.

Another object of the present invention is to provide a childbirth control system and a method thereof, in which an obstetrician can be adequately communicated with the woman in childbed, the protector thereof, and the collaborating obstetrician on the information of the progress of childbirth, whereby inducing a tailored type childbirth method through the collaborating medical treatment. Moreover, it can more stably control the childbirth through an objective and scientific childbirth management.

To accomplish the objects, the present invention provides a childbirth control system comprising: a plurality of PDAs (personal digital assistants) 10 for obtaining physical information of a woman in childbed and a fetus through a plurality of diagnosis devices electrically connected thereto in real-time so as to build a personal childbirth process information database; a childbirth server 20 for comparing the personal childbirth process information data obtained from each PDA (personal digital assistants) 10 in real-time through a wire/wireless communication network with a stored basic childbirth process information data and retransmitting the analyzed data to the corresponding PDA (personal digital assistants) 10 so as to set any one childbirth method of a natural childbirth and a artificial childbirth; and a plurality of monitoring terminals 30 for outputting the personal childbirth process information data obtained from each PDA (personal digital assistants) 10 in real-time through the wire/wireless communication network and the progress of the childbirth with a sound or image in order that the woman in childbed and collaborating obstetrician can visually make out them.

Here, the diagnosis devices comprise a heartbeat measuring instrument 11, a cervix measuring instrument 12, a lightening measuring instrument 13, an uterine contraction measuring instrument 14, an ultrasonic image device 15, and a camera 16.

The personal childbirth process information database comprises a measuring data measured by the diagnosis devices, a selection data of a childbirth method selected by the woman in childbed, and a childbirth chart for outputting the measuring data with an image according to the selection data.

The childbirth chart includes an Y-axis having a dilatation line illustrating an opening size (cm) of the womb, an X-axis illustrating an hour, four indicating lines and two section lines, four indicating lines and two section lines being varied according to the childbirth method.

The section lines comprise a vertical pang line and a vertical childbirth line.

Each indicating line comprises an optimum indicating line extending from an admission time into a hospital to a pang line, a warning indicating line, a diagnosis indicating line, and a firm diagnosis indicating line displayed between the section lines in order.

Here, the optimum indicating line, the warning indicating line, the diagnosis indicating line and the firm diagnosis indicating line are expressed by the following numerical formula 1 through 4 respectively.

Y=aX+b [Math FIG. 1]

Here, “a” is a slope and “b” is a constant.

Y=aX+c [Math FIG. 2]

Here, “a” is a slope and “c” is a constant.

Y=aX+d [Math FIG. 3]

Here, “a” is a slope and “d” is a constant.

Y=aX+e [Math FIG. 4]

Here, “a” is a slope and “e” is a constant.

The constant values of each indicating line are determined according to the personal childbirth process information database of the woman in childbed and are “b” value>“c” value>“d” value>“e” value.

The basic childbirth process information database of the childbirth server 20 comprises a basic data and a warning data expressed by comparing the basic data with the personal childbirth process information database.

To accomplish the objects, the present invention provides a childbirth control method using a child birth control system having a plurality of PDAs (personal digital assistants) for obtaining physical information of a woman in childbed and a fetus through a plurality of diagnosis devices, and a childbirth server and monitoring terminals connected to the PDAs through a wire/wireless communication network, comprising the steps of: selecting a first childbirth method by the woman in childbed and protector thereof; building a personal childbirth process information database by obtaining physical information of a woman in childbed and a fetus through a plurality of diagnosis devices electrically connected each PDA according to the selected first childbirth method; outputting the established personal childbirth process information database to each PDA with a childbirth chart image and providing the established personal childbirth process information database to the childbirth server and the monitoring terminal; comparing the personal childbirth process information database obtained from each PDA (personal digital assistants) in real-time with the stored basic childbirth process information database and retransmitting the analyzed output data to the corresponding PDA (personal digital assistants); and inducing a second childbirth method to the woman in childbed and the protector through the reorganized childbirth chart in the PDA and monitoring terminal according to the retransmitted output data.

Here, the first childbirth method comprises a natural childbirth and an artificial childbirth.

In the retransmitted step of the analyzed output data, the childbirth server severs to treat a member registration by using the personal childbirth process information database of each PDA (personal digital assistants) to establish a member database.

The second childbirth method comprises a natural childbirth, a positive childbirth, a selection childbirth, a corresponding childbirth and an emergency childbirth between the natural childbirth and artificial childbirth.

The collaborating obstetrician outputs an opinion of the childbirth treatment to the corresponding PDA through sound/image information thereof.

Each collaborating obstetrician is communicated with each other on the opinion of the childbirth treatment through sound/image information of at least two monitoring terminals.

### BRIEF DESCRIPTION OF THE DRAWINGS

The above as well as the other objects, features and advantages of the present invention will be more apparent from the following detailed description taken in conjunction with the accompanying drawings, in which:

FIG. 1 is a block diagram illustrating a childbirth control system according to the present invention;

FIG. 2 is a flow chart illustrating a childbirth control system according to the present invention;

FIG. 3 is a conceptual view illustrating a childbirth chart of a childbirth control system according to the present invention;

FIG. 4 is a conceptual view illustrating a childbirth chart of a childbirth control system according to one embodiment of the present invention;

FIG. 5 is a conceptual view illustrating a childbirth chart of a childbirth control system according to another embodiment of the present invention;

FIG. 6 is a flow chart illustrating a childbirth control method according to the present invention;

FIG. 7 is a detailed flow chart illustrating a step 400 of FIG. 6; and

FIG. 8 is a flow chart illustrating a childbirth control method according to FIG. 6.

### DETAILED DESCRIPTION OF THE INVENTION

A preferred embodiment of the invention will be described in detail below with reference to the accompanying drawings.

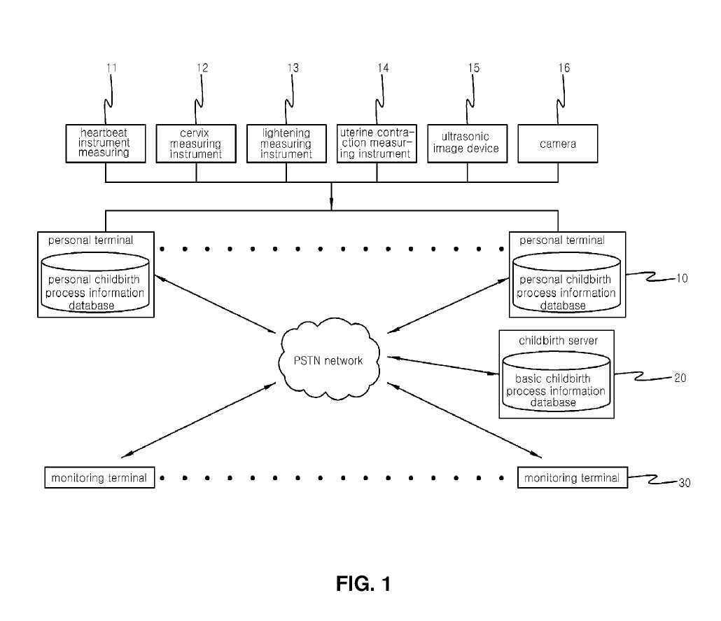

FIG. 1 is a block diagram illustrating a childbirth control system according to the present invention.

As shown in FIG. 1, the present invention can give a remote medical treatment in real-time through a communication network and can select a childbirth method according to a physical change of the woman in childbed and the fetus. Also, the obstetrician can be adequately communicated with the woman in childbed, the protector thereof, and the collaborating obstetrician on the information of the progress of childbirth, thereby inducing a tailored type childbirth method through the collaborating medical treatment. Moreover, it can more stably control the childbirth through an objective and scientific childbirth management.

The childbirth control system includes broadly three parts, that is, a plurality of PDAs (personal digital assistants) 10, a childbirth server 20, and a plurality of monitoring terminals 20. Here, they are connected to each other through the wire/wireless communication network.

Here, each PDA (personal digital assistants) 10 can obtain the physical information of a woman in childbed and a fetus through a plurality of diagnosis devices electrically connected thereto in real-time so as to build a personal childbirth process information database.

The plurality of diagnosis devices can include a heartbeat measuring instrument 11, a cervix measuring instrument 12, a lightening measuring instrument 13, an uterine contraction measuring instrument 14, an ultrasonic image device 15, and a camera 16. However, various measuring instruments can be connected thereto according to the emergency and unforeseen circumstances. The present invention is not limited to the kind of the measuring instruments.

Also, the personal childbirth process information database includes a measuring data measured by the diagnosis devices, a selection data of a childbirth method selected by the woman in childbed, and a childbirth chart for outputting the measuring data with an image according to the selection data.

That is, in the personal childbirth process information database, when the woman in childbed selects the selection data of the childbirth method, the measuring data is inputted to the childbirth chart of the corresponding childbirth method.

Accordingly, the woman in childbed, protector, and obstetrician can check the corresponding childbirth chart through a graph in real-time according to the childbirth method selected by the woman in childbed.

In the meantime, the childbirth chart includes four indicating lines and two section lines in order to confirm the entire healthy (physical) information of the woman in childbed once for all. Here, the healthy information of the fetus can be displayed in the childbirth chart by a sheet, a graph or a numerical value.

Accordingly, the pangs of childbirth, childbirth and emergency circumstances by time can be predicted through the four indicating lines and two section lines.

Also, the childbirth server 20 severs to compare the personal childbirth process information database obtained from each PDA (personal digital assistants) 10 in real-time through the wire/wireless communication network with the stored basic childbirth process information database and retransmit the analyzed data to the corresponding PDA (personal digital assistants) 10 so as to set the childbirth method.

Here, the basic childbirth process information database includes a basic data and a warning data expressed by comparing the basic data with the personal childbirth process information database.

That is, the basic data is various statistical data values including average values of the accumulated personal childbirth process information database. Accordingly, in the childbirth server 20, the personal childbirth process information database obtained from each PDA (personal digital assistants) 10 in real-time is compared with the basic data, so that it transmits the warning data to the corresponding PDA (personal digital assistants) 10 according to the variable thereof, thereby coping with the emergency circumstances.

Here, the warning data may a sound, an image or a combined data thereof.

Therefore, as though the woman in childbed selects a natural childbirth, an artificial labor can forcibly induced by means of the childbirth server 20 during the emergency and unforeseen circumstances.

Also, each monitoring terminal 30 severs to output the personal childbirth process information database obtained from each PDA (personal digital assistants) 10 in real-time through the wire/wireless communication network and the progress of the childbirth with a sound or image in order that the obstetrician can visually make out them.

Accordingly, each monitoring terminal 30 can receive a part or all of the personal childbirth process information database transmitted from each PDA (personal digital assistants) 10 to the childbirth server 20.

That is, each monitoring terminal 30 can receive the physical change per an hour or the physical diagonal information of the woman in childbed and the fetus in real-time image, the collaborating obstetrician of the corresponding monitoring terminal 30 can be adequately communicated with the corresponding obstetrician of the PDA (personal digital assistants) 10 on the information of the progress of childbirth, thereby inducing a tailored type childbirth method through the collaborating medical treatment.

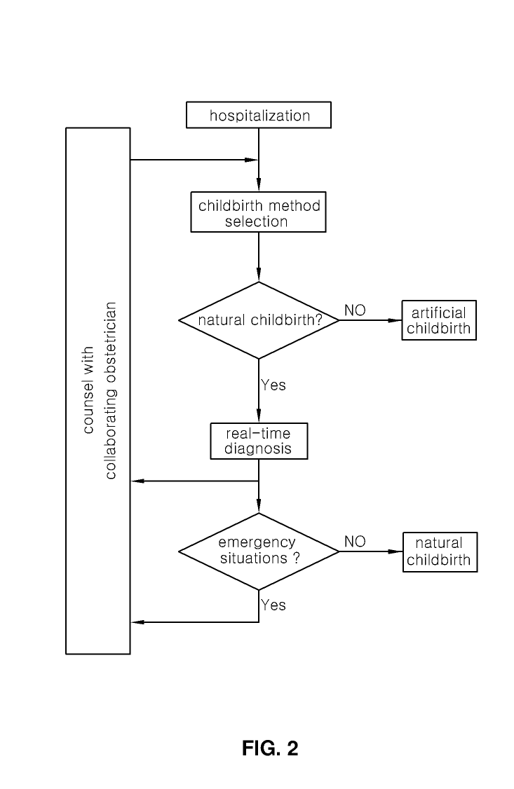

FIG. 2 is a flow chart illustrating a childbirth control system according to the present invention.

As shown in FIG. 2, firstly, the woman in childbed enters hospital for hers childbirth.

Then, the woman childbed can select the natural childbirth or artificial childbirth.

Next to the selection, the physical diagonal information of the woman in childbed and the fetus is transmitted to the PDAs (personal digital assistants) 10 of the woman in childbed, protector, and obstetrician connected to the diagnosis devices through the childbirth chart of the corresponding childbirth method in real-time.

In this case, the PDAs (personal digital assistants) 10 are connected to the monitoring terminal 30 of the collaborating obstetrician and the childbirth server 20 having the basic childbirth process information through the wire/wireless communication network.

Here, in case that the woman in childbed selects the natural childbirth and the measuring values transmitted through the PDA (personal digital assistants) 10 in real-time are different, the warning data such as the sound and image is expressed through the PDA (personal digital assistants) 10.

At this time, the collaborating obstetrician can give a remote medical treatment through the monitoring terminal 30. Also, the obstetrician can be communicated with another collaborating obstetrician as well as the woman in childbed and the protector thereof.

Accordingly, as though the woman in childbed wants the natural childbirth, the artificial labor can be induced according to the physical diagonal information of the woman in childbed and the fetus measured by the diagnosis devices in real-time.

Here, the present invention is not limited to the obstetrics and gynecology. It can be applied to various medical fields such as a dental surgery, orthopedics, and internal medicine and so on.

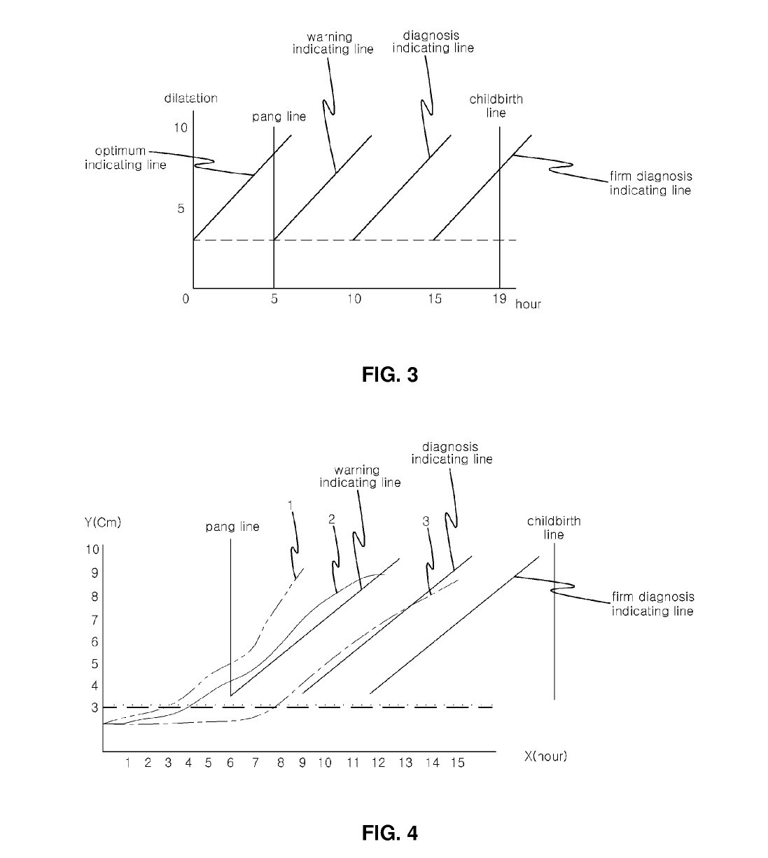

FIG. 3 is a conceptual view illustrating a childbirth chart of a childbirth control system according to the present invention.

As shown in FIG. 3, the childbirth chart includes an Y-axis having a dilatation line illustrating an opening size (cm) of the womb, an X-axis illustrating an hour, four indicating lines and two section lines.

Here, two section lines include a vertical pang line and a vertical childbirth line.

The vertical pang line is a point of time (generally 1 cm/1 hour) of actively starting the progress of the childbirth and an indicating line illustrating the progress state of about 3-5 cm. Here, the progress of the childbirth can be evaluated with the passage of time. That is, the opening size of the womb and the passage of time can be set in advance, for example 3 cm to 6 hours, 4 cm to 8 hours, and 5 cm to 10 hours. Where the setting time is exceeded, it diagnoses the pang line as an abnormal state.

The childbirth line is an indicating line illustrating the passage of time of 1-4 hours from a full dilatation (about 10 cm) to a delivery of a newborn baby. Here, the progress of the childbirth line can be evaluated with the passage of time. That is, the woman who has had her first childbirth corresponds to about 2 hours and the multiparous woman corresponds to about 1 hour. Where the setting time is exceeded, it diagnoses the childbirth line as an abnormal state.

In the meantime, four indicating lines includes an optimum indicating line, a warning indicating line, a diagnosis indicating line, and a firm diagnosis indicating line. Here, each indicating line can be evaluated according to the speed of the childbirth (dilatation and lightening) and yes or no of the passing of the indicating line.

Also, four indicating lines include the optimum indicating line extending from the admission time into a hospital to the pang line, the warning indicating line, diagnosis indicating line, and firm diagnosis indicating line displayed between the section lines in order.

Here, the optimum indicating line as a properly basic childbirth standard corresponds to the speed of the dilatation of 1 cm per 1 hour. For example, where the childbirth progress line generated by the measuring data according to the passage of the childbirth passes through the optimum indicating line established by the childbirth chart based on the basic data, it diagnoses the childbirth progress line as an abnormal childbirth. Accordingly, in order to give birth to a baby within the range of the optimum indicating line, it is necessary to accompany an active medical treatment. That is, in case that the predetermined range (setting time) set by the optimum indicating line is exceeded, the medical treatment including a caesarean operation should be required.

Here, the warning indicating line corresponds to the speed of the dilatation of 1 cm per 1 hour from the pang line and is a standard indicating line on the presumption of a childbirth obstacle.

Also, the diagnosis indicating line is an indicating line illustrating a state progressed for 1 through 8 hours from the warning indicating line according to the childbirth method and is a standard indicating line on the presumption of a childbirth obstacle and yes or no of the medical treatment according to the childbirth obstacle.

Moreover, the firm diagnosis indicating line is an indicating line illustrating a state progressed for 1 through 8 hours from the diagnosis indicating line according to the childbirth method and is a standard indicating line on the firm diagnosis of the childbirth obstacle.

It is preferred that the above indicating lines are expressed by the following numerical formula 1 through 4.

Y=aX+b (optimum indicating line) [Math FIG. 1]

Here, “a” is a slope and “b” is a constant.

Y=aX+c (warning indicating line) [Math FIG. 2]

Here, “a” is a slope and “c” is a constant.

Y=aX+d (diagnosis indicating line) [Math FIG. 3]

Here, “a” is a slope and “d” is a constant.

Y=aX+e (firm diagnosis indicating line) [Math FIG. 4]

Here, “a” is a slope and “e” is a constant.

The slope value “a” is a constant obtained by an experiment and experience. For example, the average value calculated by the inspection of the state of the woman in childbed may be the basic childbirth process information database.

Here, the constant values of each indicating line are determined according to the personal childbirth process information database of the woman in childbed. Here, preferably, it is “b” value>“c” value>“d” value>“e” value.

It is axiomatic that the locations of four indicating lines and two section lines can be varied according to the personal childbirth process information database measured by each woman in childbed.

The childbirth chart will be briefly described below with reference to the accompanying drawings.

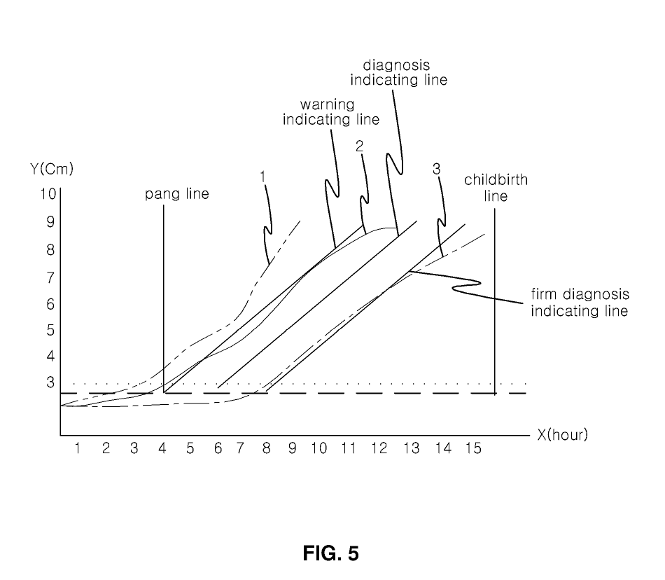

FIG. 4 is a conceptual view illustrating a childbirth chart of a childbirth control system according to one embodiment of the present invention and FIG. 5 is a conceptual view illustrating a childbirth chart of a childbirth control system according to another embodiment of the present invention.

Here, since the optimum indicating line indicates a normal childbirth, it is omitted here.

As shown in FIG. 4, in the childbirth chart, three indicating lines and two section lines are established in such a manner that the natural childbirth can be allowable to the maximum.

Firstly, line 1 illustrates the opening speed of the dilatation with the pang from the admission time into a hospital with the passage of time. As shown, the dilatation becomes open to 10 cm without being reached to the warning indicating line. Accordingly, it can confirm the normal natural childbirth.

Also, line 2 extends to the warning indicating line and indicates the normal childbirth. However, since it is estimated to be a childbirth obstacle, the obstetrician must act with prudence.

In line 3, the dilatation becomes open after 2-3 hours from the pang line. Also, since the dilatation becomes open next to 14-15 hours from the admission time into a hospital enough to give a birth to a child, it diagnoses line 3 as a childbirth obstacle. Accordingly, it can be determined as to whether the medical treatment is performed or not owing to the childbirth obstacle. Here, after the medical treatment, where line 3 extends to the firm diagnosis indicating line, the childbirth obstacle is confirmed. In this case, the expediency such as a caesarean operation is required.

As shown in FIG. 5, in the childbirth chart, three indicating lines and two section lines are established in such a manner that the natural childbirth can be allowable identically with FIG. 4, not to the maximum. That is, FIG. 5 illustrates a graph for inducing a partial natural childbirth. Accordingly, the childbirth charts of FIG. 4 and FIG. 5 are different from each other in terms of the definition of the childbirth obstacle and the treatment time thereof.

As described above, the PDA (personal digital assistants) 10 or the monitoring terminal 30 according to the childbirth control system of the present invention includes an alarm of the warning indicating line (for example, direct image warning such as a red line or a red word), an alarm of the diagnosis indicating line (for example, a warning alarm and a sound warning such as a beeper), and an alarm of the firm diagnosis indicating line (for example, image, sound and monitor flickering warnings) for directly informing the dangerous situations according to the physical diagonal information of the woman in childbed (the personal childbirth process information database of the woman in childbed).

### ADVANTAGEOUS EFFECTS

As can be seen from the foregoing, in the childbirth control system and the method thereof, it can give the remote medical treatment in real-time through the communication network and select the childbirth method according to the physical change of the woman in childbed and the fetus.

Also, the obstetrician can be adequately communicated with the woman in childbed, the protector thereof, and the collaborating obstetrician on the information of the progress of childbirth, whereby inducing a tailored type childbirth method through the collaborating medical treatment. Moreover, it can more stably control the childbirth through an objective and scientific childbirth management.

### BEST MODE

A preferred embodiment of the invention will be described in detail below with reference to the accompanying drawings.

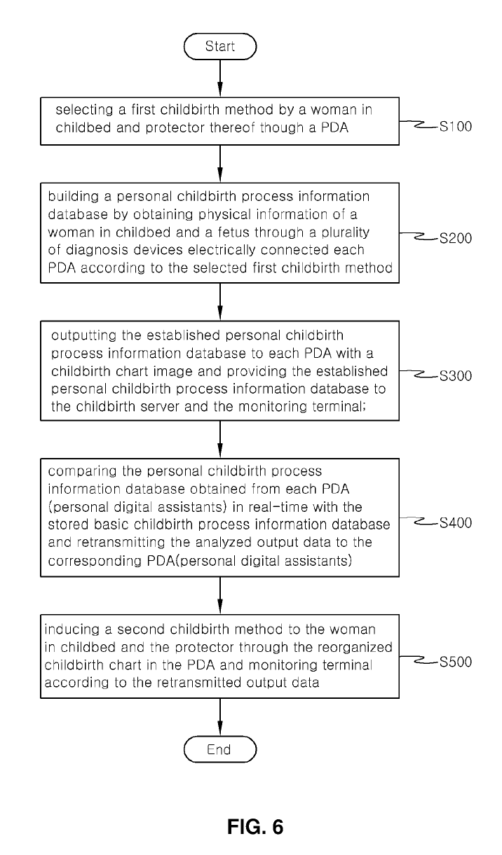

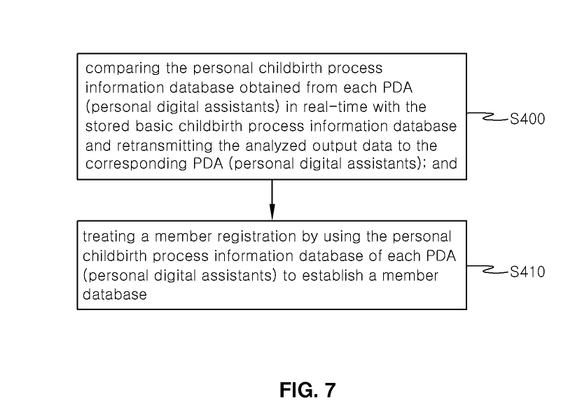

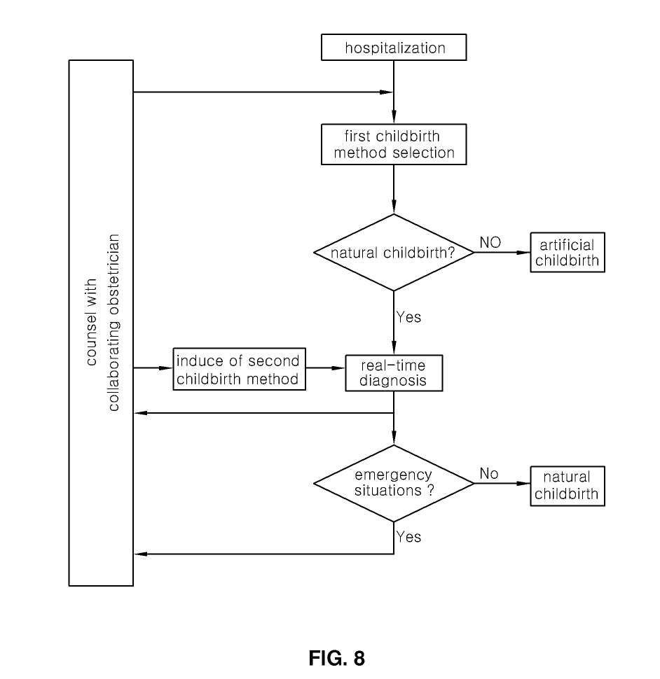

FIG. 6 is a flow chart illustrating a childbirth control method according to the present invention, FIG. 7 is a detailed flow chart illustrating a step 400 of FIG. 6, and FIG. 8 is a flow chart illustrating a childbirth control method according to FIG. 6.

As shown in FIG. 6 through FIG. 8, the present invention can give a remote medical treatment in real-time through a communication network and can select a childbirth method according to a physical change of the woman in childbed and the fetus. Also, the obstetrician can be adequately communicated with the woman in childbed, the protector thereof, and the collaborating obstetrician on the information of the progress of childbirth, thereby inducing a tailored type childbirth method through the collaborating medical treatment. Moreover, it can more stably control the childbirth through an objective and scientific childbirth management.

In the childbirth control method, the woman in childbed or the protector can select any one of the artificial labors (a positive childbirth, a selection childbirth, a corresponding childbirth, and an emergency childbirth) and the natural childbirth.

Here, basically, when the woman in childbed selects the first childbirth method, a measuring data is inputted to the childbirth chart of the corresponding childbirth method.

Firstly, various information such as the expected date of delivery of the woman in childbed, a final menstrual, a healthy condition and physical information of the woman in childbed and the fetus and so forth can be inputted through the PDA 10 (note S100).

Then, the physical information of the woman in childbed and the fetus can be obtained in real-time through the diagnosis devices connected to the PDA 10 according to the selected first childbirth method to establish the personal childbirth process information database (note S200).

Continuously, the PDA 10 allows the established personal childbirth process information database to be outputted with a chart image and the woman in childbed, protector and obstetrician can visually check the corresponding childbirth chart in order to be communicated with each other.

Also, the PDA 10 allows the obtained personal childbirth process information database to provide to the childbirth server 20 and the monitoring terminal 30 through the communication network.

Here, the PDA 10, the childbirth server 20 and the monitoring terminal 30 should be permissible. That is, only permissible terminals can receive the personal childbirth process information database. Accordingly, each collaborating obstetrician can be communicated with each other on the opinion of the childbirth treatment through sound/image information of the monitoring terminals.

Accordingly, the collaborating obstetrician of the corresponding monitoring terminal 30 can be adequately communicated with the corresponding obstetrician of the PDA (personal digital assistants) 10 on the opinion of the childbirth treatment through the sound/image information (S300).

Then, the childbirth server 20 severs to compare the personal childbirth process information database obtained from each PDA (personal digital assistants) 10 in real-time through the wire/wireless communication network with the stored basic childbirth process information database and retransmit the analyzed data to the corresponding PDA (personal digital assistants) 10 (S400). Here, the childbirth server 20 severs to treat a member registration by using the personal childbirth process information database of each PDA (personal digital assistants) 10 to establish a member database (S410).

Continuously, a second childbirth method can be induced to the woman in childbed and the protector thereof through the reorganized childbirth chart in the PDA 10 according to the retransmitted output data. That is, when the emergency situations are occurred through the warning data, the childbirth chart in the PDA 10 is reconstructed to be outputted, so that the corresponding obstetrician can cope with the emergency situations and the second childbirth method can be induced to the woman in childbed and the protector.

Here the second childbirth method includes the natural childbirth, positive childbirth, selection childbirth, corresponding childbirth and emergency childbirth between the natural childbirth and artificial childbirth.

Also, each monitoring terminal 30 can receive a part or all of the personal childbirth process information database transmitted from each PDA (personal digital assistants) 10 to the childbirth server 20, so that the obstetrician can be adequately communicated with the collaborating obstetrician on the information of the progress of childbirth, thereby inducing the tailored type childbirth method through the collaborating medical treatment.

Here, the present invention is not limited to the obstetrics and gynecology. It can be applied to various medical fields such as a dental surgery, orthopedics, and internal medicine and so on.

### INDUSTRIAL APPLICABILITY

The present invention relates to a childbirth control system and a method thereof, and more particularly to a childbirth control system and a method thereof capable of giving a remote medical treatment in real-time through a communication network and selecting a childbirth method according to a physical change of the woman in childbed and the fetus.

While this invention has been described in connection with what are presently considered to be the most practical and preferred embodiments, it is to be understood that the invention is not limited to the disclosed embodiments and the drawings, but, on the contrary, it is intended to cover various modifications and variations within the spirit and scope of the appended claims.

1. A childbirth control system comprising:
a plurality of PDAs (personal digital assistants) for obtaining physical information of a woman in childbed and a fetus through a plurality of diagnosis devices electrically connected thereto in real-time so as to build a personal childbirth process information database;
a childbirth server for comparing the personal childbirth process information data obtained from each PDA (personal digital assistants) in real-time through a wire/wireless communication network with a stored basic childbirth process information data and retransmitting the analyzed data to the corresponding PDA (personal digital assistants) so as to set any one childbirth method of a natural childbirth and a artificial childbirth; and
a plurality of monitoring terminals for outputting the personal childbirth process information data obtained from each PDA (personal digital assistants) in real-time through the wire/wireless communication network and the progress of the childbirth with a sound or image in order that the woman in childbed and collaborating obstetrician can visually make out them.

2. A childbirth control system as claimed in claim 1, wherein the diagnosis devices comprise a heartbeat measuring instrument, a cervix measuring instrument, a lightening measuring instrument, an uterine contraction measuring instrument, an ultrasonic image device, and a camera.

3. A childbirth control system as claimed in claim 1, wherein the personal childbirth process information database comprises a measuring data measured by the diagnosis devices, a selection data of a childbirth method selected by the woman in childbed, and a childbirth chart for outputting the measuring data with an image according to the selection data.

4. A childbirth control system as claimed in claim 3, wherein the childbirth chart includes an Y-axis having a dilatation line illustrating an opening size of the womb, an X-axis illustrating an hour, four indicating lines and two section lines, four indicating lines and two section lines being varied according to the childbirth method.

5. A childbirth control system as claimed in claim 3, wherein the section lines comprise a vertical pang line and a vertical childbirth line.

6. A childbirth control system as claimed in claim 3, wherein each indicating line comprises an optimum indicating line extending from an admission time into a hospital to a pang line, a warning indicating line, a diagnosis indicating line, and a firm diagnosis indicating line displayed between the section lines in order.

7. A childbirth control system as claimed in claim 6, wherein the optimum indicating line, the warning indicating line, the diagnosis indicating line and the firm diagnosis indicating line are expressed by the following numerical formula 1 through 4, respectively:
Y=aX+b; <Formula 1>
wherein “a” is a slope and “b” is a constant;
Y=aX+c; <Formula 2>
wherein “a” is a slope and “c” is a constant;
Y=aX+d; <Formula 3>
wherein “a” is a slope and “d” is a constant;
Y=aX+e; <Formula 4>
wherein “a” is a slope and “e” is a constant.

8. A childbirth control system as claimed in claim 7, wherein the constant values of each indicating line are determined according to the personal childbirth process information database of the woman in childbed and are “b” value>“c” value>“d” value>“e” value.

9. A childbirth control system as claimed in claim 1, wherein the basic childbirth process information database of the childbirth servers comprises a basic data and a warning data expressed by comparing the basic data with the personal childbirth process information database.

10. A childbirth control method using a child birth control system having a plurality of PDAs (personal digital assistants) for obtaining physical information of a woman in childbed and a fetus through a plurality of diagnosis devices, and a childbirth server and monitoring terminals connected to the PDAs through a wire/wireless communication network, comprising the steps of:
selecting a first childbirth method by the woman in childbed and protector thereof;
building a personal childbirth process information database by obtaining physical information of a woman in childbed and a fetus through a plurality of diagnosis devices electrically connected each PDA according to the selected first childbirth method;
outputting the established personal childbirth process information database to each PDA with a childbirth chart image and providing the established personal childbirth process information database to the childbirth server and the monitoring terminal;
comparing the personal childbirth process information database obtained from each PDA (personal digital assistants) in real-time with the stored basic childbirth process information database and retransmitting the analyzed output data to the corresponding PDA (personal digital assistants); and
inducing a second childbirth method to the woman in childbed and the protector through the reorganized childbirth chart in the PDA and monitoring terminal according to the retransmitted output data.

11. A childbirth control method as claimed in claim 10, wherein the first childbirth method comprises a natural childbirth and an artificial childbirth.

12. A childbirth control method as claimed in claim 10, wherein in the retransmitted step of the analyzed output data, the childbirth server severs to treat a member registration by using the personal childbirth process information database of each PDA (personal digital assistants) to establish a member database.

13. A childbirth control method as claimed in claim 10, wherein the second childbirth method comprises a natural childbirth, a positive childbirth, a selection childbirth, a corresponding childbirth and an emergency childbirth between the natural childbirth and artificial childbirth.

14. A childbirth control method as claimed in claim 10, wherein the collaborating obstetrician outputs an opinion of the childbirth treatment to the corresponding PDA through sound/image information thereof.

15. A childbirth control method as claimed in claim 10, wherein each collaborating obstetrician is communicated with each other on the opinion of the childbirth treatment through sound/image information of at least two monitoring terminals.
